(19) United States
(12) Patent Application Publication
(10) Pub. No.: US 2025/0075252 A1
(43) Pub. Date: Mar. 6, 2025

(54) SAMPLE TESTING DEVICES AND METHODS

(71) Applicant: Gate Scientific, Inc., Milpitas, CA (US)

(72) Inventors: Kristian Michael SCABOO, Castro Valley, CA (US); Morten Juel JENSEN, Saratoga, CA (US); Jesse OROZCO, Sunnyvale, CA (US)

(73) Assignee: Gate Scientific, Inc., Milpitas, CA (US)

(21) Appl. No.: 18/806,579

(22) Filed: Aug. 15, 2024

Related U.S. Application Data
(63) Continuation of application No. PCT/US2023/062686, filed on Feb. 15, 2023.
(60) Provisional application No. 63/268,047, filed on Feb. 15, 2022.

(51) Int. Cl. C12Q 1/6806; B01L 7/00; C12Q 1/6844

(52) U.S. Cl. CPC C12Q 1/6806; B01L 7/52; C12Q 1/6844

(57) ABSTRACT

A method for separating and analyzing a liquid sample, such as a blood sample, is disclosed. The method may include combining the sample with liquid reagents, then heating the sample to an appropriate temperature for a sufficient time. The mixed sample and reagents May then be passed through a filter. The resulting liquid may then be exposed to light in a detection chamber and detected by an optical sensor. The data output from the optical sensor may be shown on a display on the device and/or transmitted wirelessly or over a wire to another device. A device for performing the method is also disclosed.

### CROSS-REFERENCE TO RELATED APPLICATION

This application is a continuation of International Application No. PCT/US2023/062686, filed Feb. 15, 2023, which claims priority to U.S. Provisional Application No. 63/268,047, filed 15 Feb. 2022, both of which are incorporated by reference herein in their entireties.

### BACKGROUND

Blood naturally contains nucleic acid amplification inhibitors. It is desired to separate the nucleic acids in blood from the inhibitors so the nucleic acid can be amplified and analyzed (e.g., with PCR, isothermal, and/or other amplification methods).

Typically to perform this separation, blood samples are put in a lysis/binding solution to lyse the blood and make it bind to a separating component, for example magnetic beads and resins of silica.

This lysing/binding solution lyses intact cells from the blood sample, releasing nucleic acid. Typically, lysing solutions are not compatible with amplification procedures. The nucleic acid must be purified from the lysing solution. For example, nucleic acid binds to silica with magnetic beads which then binds to a solid magnetic surface. The unbound inhibitors and other blood components and solution are then washed with alcohol, generally leaving the nucleic acid bound to the silica. The bound nucleic acid is then eluted from the silica with an aqueous buffer.

This results in a separation of the nucleic acid. However, these steps often inhibit amplification (e.g., PCR), and the alcohol that is used for washing is volatile. Also, each wash step results in more steps, expenses, and losses of the sample. This process just to isolate the nucleic acid for one sample often takes 30-60 minutes. The magnetic (or other) binding also isn't 100% effective, so some target nucleic acid gets washed away. These issues make it difficult, time consuming, and expensive to automate the process and incorporate into a user-friendly device.

Accordingly, a simpler and more effective separation method and accompanying device is desired.

### SUMMARY

Methods for separating components of a liquid sample are disclosed. In some embodiments, the methods may include mixing the liquid sample with liquid reagents. In some embodiments, the liquid reagents may include additives for removing inhibitors from the blood sample, reducing secondary structure of nucleic acids, and adding components necessary for amplification. These additives may include monovalent cations, divalent cations including barium, copper [II}, calcium, magnesium, manganese [II], zinc, iron [II], nickel, cobalt, Tin [II], cadmium, lead, multivalent cations, anions including sulfate and chloride, naturally occurring or modified amino acids, oligonucleotides, buffer components, and any other additives to improve the amplification yield. In some embodiments, the additives may include magnesium and betaine. The methods may include heating the mixed liquid sample and liquid reagents to 70° C. to 120° C. for 10 seconds to 20 minutes, more narrowly for 5-10 minutes at 80° C. to 99° C., or even more narrowly for 5-10 minutes at 85-95° C. These methods may include causing a precipitate to form when the mixed liquid sample and liquid reagents are heated. The methods may include centrifuging the heated mixed liquid sample and liquid reagents. The methods may include forcing the heated mixed liquid sample and liquid reagents through a filter. The method may include liquid reagents that are mixed with the liquid sample, are heated with the liquid sample 17 causing a precipitate to form, are still present in the supernatant after removing the precipitate, and are used in a subsequent nucleic acid amplification reaction without any further purification, dilution, or treatment. The method may include exposing the supernatant or filtered solution to lyophilized or dried reagents that when reconstituted, participate in a nucleic acid amplification. The nucleic acid amplification method may be isothermal, use multiple thermal steps, or use thermocycling protocols. The nucleic acid amplification method may be strand displacement amplification (SDA). In some embodiments a nucleic acid reporter oligonucleotide containing a detectable moiety is present during the amplification reaction. In some embodiments a nucleic acid reporter oligonucleotide containing a custom nucleic acid sequence region may be used for detection.

The liquid sample may be a blood sample. The liquid sample could be the eluent from a swab. The swab sample could be oral, nasal, nasopharyngeal, throat, mouth, check, skin, a lesion, rectal, fecal, vaginal, urethral. The swab sample could be environmental. The methods may include exposing the resulting filtered liquid to a controlled light frequency and sensing a light frequency emitted, absorbed or transmitted from the resulting filtered liquid. In some embodiments a nucleic acid reporter oligonucleotide containing a zip code region containing a custom nucleic acid sequence region may be used for detection.

Methods, devices and reagents for separating components of a liquid sample are disclosed. The devices may have a case holding a liquid reagent chamber, an inlet port for receiving the liquid sample, a processing chamber for mixing and heating the liquid sample and reagents, a processing chamber heater, a processing chamber exit valve, a processed sample filter, a detection chamber, an optical sensor adjacent to the detection chamber; and a light emitter adjacent to the detection chamber. Part or all of the detection chamber may be transparent. The liquid reagent chamber may be at least partially or completely filled with a liquid reagent comprising magnesium or betaine or both. The devices may have a gas-permeable membrane on the detection chamber to allow for liquid delivery.

The device may be a self-contained device that has all components implemented to perform an analytical test including detection, heating, sensing, computing, power source, display and/or wireless transmission. The device may contain one or more circuit boards or flex boards or combinations thereof.

The device may be powered from batteries or by solar power or by a USB connection or wireless power induction or by any external power source.

A nucleic acid reporter oligonucleotide that may be added to an amplification reaction to allow for multiplexed detection of nucleic acid targets is disclosed. In some embodiments, this reporter oligonucleotide may contain a zip code region labeled with a detectable tag that is complementary to an oligonucleotide capture probe that may be exposed to the reaction. During the course of the reaction, this single stranded zip code sequence may be released from the reporter molecule in the presence of the desired target and may then be available to hybridize to the oligonucleotide capture probe and be detected or form another complex to initiate another event or detection mechanism. The oligonucleotide capture probe could be on a surface or in solution. The surface could be flat or spherical. The capture probes could be in an array of capture probes. The complex could be an oligonucleotide-oligonucleotide complex, where one or more of the oligos could contain non-naturally occurring nucleotides. The complex could be an oligonucleotide-protein complex, where the protein could be an enzyme or a non-enzymatic protein. The oligonucleotide-protein complex could contain double or single stranded oligonucleotides. The complexes could initiate other biochemistry or reporter processes such as strand specific cleavage of a nucleic acid, or enzymatic signal amplification. The complexes could initiate a nucleic acid amplification event. The label on the zip code region may be detected using low cost, low power methods.

A method for separating components of a liquid sample id disclosed. The method can include mixing the liquid sample with reagents, heating the mixed liquid sample and liquid reagents from about 80° C. to about 99° C., and separating the resulting precipitate from the liquid supernatant. The reagents can have a divalent cation. The reagents can be or have liquid reagents. The reagents can be or have dried reagents. The precipitate can be separated from the liquid supernatant by centrifugation. The precipitate can be separated from the liquid supernatant by forcing the heated mixed liquid sample and liquid reagents through a filter. The liquid sample can have a blood sample. The divalent cation can be magnesium. The liquid reagents can have betaine. The heating can last for at least 10 seconds. The heating can last for at least or exactly 5 minutes at about 95° C.

The method can include exposing the resulting filtered liquid to a controlled light frequency and sensing a light frequency emitted, absorbed, or transmitted from the resulting filtered liquid.

The resulting precipitate can remove nucleic acid inhibitors from the liquid supernatant. The method can include performing a nucleic acid amplification on the liquid supernatant. The method can include detecting the presence of a nucleic acid by fluorescence, absorbance, electrochemical detection, or combinations thereof. The method can include performing a nucleic acid amplification on the liquid supernatant without further dilution.

A device for separating components of a liquid sample is disclosed. The device can have a reagent chamber containing reagents, an inlet port for receiving the liquid sample, a processing chamber, a processing chamber heater, a processing chamber exit valve, a processed sample filter, a detection chamber, an optical sensor adjacent to the detection chamber, or combinations thereof. The reagents can have a divalent cation. The detection chamber can be at least partially transparent. The device can include a light emitter adjacent to the detection chamber. The liquid sample can have a blood sample. The divalent cation can be magnesium. The liquid reagents can have betaine. The heating can last for at least 10 seconds. The device can have a detection chamber membrane on the detection chamber. The reagents can be liquid reagents. The reagents can be dried reagents.

A system for separating components of a liquid sample is disclosed. The system can have a reagent chamber containing reagents, an inlet port for receiving the liquid sample, a processing chamber, a processing chamber heater, a processing chamber exit valve, a processed sample filter, a detection chamber, an optical sensor adjacent to the detection chamber, a light emitter adjacent to the detection chamber, or combinations thereof. The reagents can have a divalent cation. The detection chamber can be at least partially transparent.

The liquid sample can have a blood sample. The divalent cation can be magnesium. The liquid reagents can have betaine. The heating can last for at least 10 seconds.

The system can have a detection chamber membrane on the detection chamber. The reagents can be or have liquid reagents. The reagents can be or have dried reagents.

A reporter oligonucleotide is disclosed. The reporter oligonucleotide can indicate the presence of a target nucleic acid in a sample where a portion of a reporter oligonucleotide gains the ability to hybridize to a non-target specific oligonucleotide capture probe as a result of the presence of the target. The reporter oligonucleotide can contain a sequence specific to the target nucleic acid, a recognition sequence for a restriction enzyme, a polymerase extension blocker, a sequence specific to an oligonucleotide, and combinations thereof. The reporter oligonucleotide can have a secondary structure to prevent hybridization to the oligonucleotide capture probe when not in the presence of the target. The portion of the reporter oligonucleotide that is complementary to the oligonucleotide capture probe can be cleaved from the reporter oligo by the reaction of the restriction enzyme.

The oligonucleotide capture probe can be attached to an electrochemical detection surface. The presence of the hybridized portion of the reporter oligonucleotide can be detected electrochemically. A portion of the reporter oligonucleotide has a tag that can be detected using any of fluorescence, fluorescent lifetime, chemiluminescence, colorimetrically, gravimetrically, cantilever methods, surface plasmon resonance, or combinations thereof.

The oligonucleotide capture probe can be attached to a surface. The oligonucleotide capture probe can be attached to a bead. The oligonucleotide capture probe can be suspended in solution. The reporter oligonucleotide can be a reactant in a nucleic acid amplification reaction in the presence of the target.

The reporter oligonucleotide can be a reactant in an isothermal nucleic acid amplification reaction in the presence of the target. The isothermal nucleic acid amplification can have thermal steps that bring the temperature to levels different from where the isothermal reaction happens. The reporter oligonucleotide can be a reactant in a nucleic acid amplification reaction in the presence of the target.

A system is disclosed for detecting multiple nucleic acid sequences in a chamber by having the result of individual sequences hybridize to specific locations on a surface in the container. The individual sequence strands or representations of the strands can have a fluorescent tag that is measured using fluorescent lifetime.

A system is disclosed for detecting multiple nucleic acid sequences in a chamber by having the result of individual sequences hybridize to one or more beads in the container. The individual sequence strands or representations of the strands can have a fluorescent tag that is measured using fluorescent lifetime.

A system is disclosed for detecting fluorescence in a chamber by a fluorescent tag that is measured using fluorescent lifetime.

### BRIEF DESCRIPTION OF THE DRAWINGS

FIG. 1 illustrates a variation of the device.

FIG. 2 illustrates a variation of a micropipette.

FIG. 3A and FIG. 3B are computer aided design and schematic cross-sectional views, respectively, of a portion of a variation of the device.

FIG. 3C is a perspective view of a portion of the device with the cap open.

FIG. 4A and FIG. 4B are partially see-through side and top views, respectively, or a portion of a variation of the device with the cap open.

FIG. 4C is a perspective view of a portion of a variation of the device with the cap open.

FIG. 5A and FIG. 5B are partially see-through side and top views, respectively, or a portion of a variation of the device of FIG. 4A and FIG. 4B with the cap closed.

FIG. 6A is a perspective view of the detection system.

FIG. 6B is a close-up view of a portion of a variation of the fluidic system assembly.

FIG. 7 is a schematic cross-sectional view of a variation of the device.

FIG. 8 is a schematic cross-sectional view of a variation of the device.

FIG. 9 is a schematic cross-sectional view of a variation of the device.

FIG. 10 is a schematic cross-sectional view of a variation of the device.

FIG. 11 is a schematic cross-sectional view of a variation of the device.

FIG. 12 is a schematic cross-sectional view of a variation of the device.

FIG. 13 shows a flowchart outlining a typical assay for a whole blood sample.

FIG. 14A and FIG. 14B show possible control oligonucleotide configurations.

FIG. 15A and FIG. 15B show the dependance of the precipitation reaction on the magnesium concentration.

FIG. 16 shows a variation of the system in which heating, detection and fluid operation are performed by an instrument external to the device.

FIG. 17 shows a variation of a locking cap for the input port.

FIG. 18 shows a variation of the system where an external collection vial is used to load a sample into the device.

FIG. 19A and FIG. 19B show variations of the device where liquid reagents are present in the input port.

FIG. 20 shows a variation of the system where the sample is loaded from a swab.

FIG. 21 shows a variation of the device with multiple detection chambers.

FIG. 22A and FIG. 22B show examples of devices designed to use centrifugation for fluid manipulation.

FIG. 23 shows an example of a pierceable membrane valve.

FIG. 24 shows the general structure of the reporter oligonucleotide.

FIG. 25 shows an epifluorescent detection setup.

FIG. 26 shows a time-resolved fluorescent detection setup.

FIG. 27 shows a more compact, lower cost, time-resolved fluorescent detection setup.

FIG. 28 shows the most simplified time-resolved fluorescent detection setup.

FIG. 29 shows a time diagram of the time resolved fluorescent detection step.

FIG. 30 shows an electrochemical interrogation of positive and negative amplification reactions.

### DETAILED DESCRIPTION

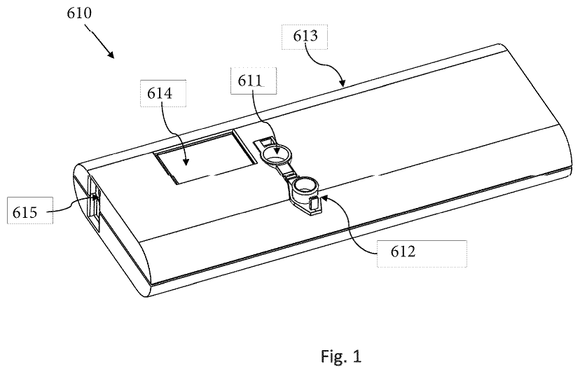

FIG. 1 illustrates an embodiment of the device 610 that has a hard case 613. The case may have openings for one or more displays 614, inlet ports 611, communication ports 615, or combinations thereof. The display may be an LCD, E-Ink, LED display, one or more LED indicators or any technology that may convey visual information.

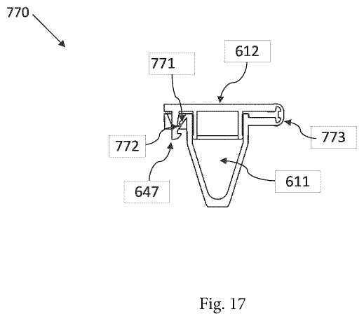

The device may have an inlet port for receiving a liquid sample to be tested. The device may have a cover, seal, lid or cap 612 for closing and scaling the inlet port. As shown in FIG. 17, the cap may be configured to activate the device (e.g., sample nucleic acid separation and analysis). The cap may be attached to and/or adjacent to the inlet port, for example by a flexible band. The cap, 612, and inlet port 611 may snap fit. The cap may have a cap closure tab 647. The case may have a cap closure port. The cap closure tab 647 may translate into the cap closure port when the cap is in a closed configuration. The cap closure tab 647 may have teeth 772 that match with a pawl feature 771 on the inlet port to prevent the cap from opening once closed. The cap closure tab 647 may press a button or switch or a feature in the cap closure port, which May mechanically activate and in turn send a signal in the device (e.g., to a processor) indicating that the cap 612 is closed and the inlet port 611 is sealed. In some embodiments, the cap 612 and inlet port 611 may be connected by a living hinge 773.

A device may have more than one inlet port 611 such that multiple samples can be processed by one device 610, for example if testing for venereal disease it may require both a swab sample and a blood sample. A device with more than one inlet port 611 may have multiple individual liquid paths with separate filters and separate membranes and separate detection chambers. A device with more than one inlet port 611 may have multiple reagent chambers or it may share one or more reagent chambers with the different inlet ports 611. A device with multiple inlet ports 611 may be configured such that the samples in the different inlet ports 611 are processed at the same time or the samples are processed at different times, for example if different persons samples are tested with one device.

The inlet port may be a capillary channel or chamber where the sample deposit is assisted by capillary force from the channel or chamber. The channel or chamber may be coated with a hydrophilic coating to assist in the deposit of a sample. The inlet port may have a cap or it May not have a cap or it may have a pierceable membrane like a rubber septum or other types of membranes. The inlet port may be under vacuum such that a sample is drawn into the fluidic structure.

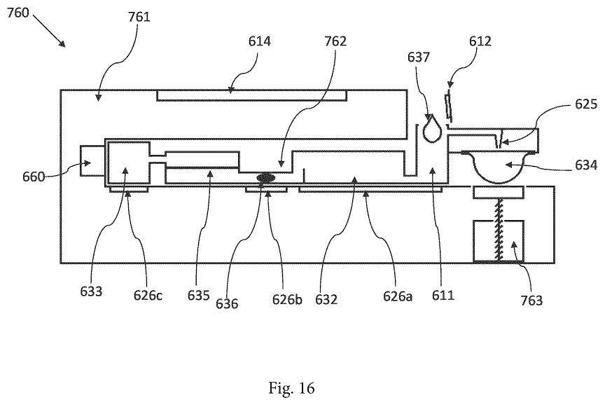

The system can consist of a disposable cartridge 762 that contains dry and liquid reagents, membranes, filters; where the disposable cartridge is mated with an instrument that can provide some or all of the processing actuations such as heating, fluid release as well as detection circuitry. Where the detection circuitry can be optical components or electrical interface to an electrochemical detection chamber in the consumable cartridge. FIG. 16 shows a exemplary device 760 where the heaters 626a, 626b, 626c, optical detection 660 and fluid actuation are in an instrument 761 along with a display 614, power source (not shown), microprocessor (not shown) and other circuitry; where the disposable cartridge 762 interfaces to the instrument and contains all dry and liquid reagents, filter(s) and, membrane(s). A valve may not be needed if the liquid reagents are released in a controlled manner for example if the liquid reagents are released by an actuator 763 that can be controlled to provide a specific amount of fluid in certain sequences.

The communication port may have one or more plug outlets and/or wireless (e.g., Bluetooth, Wifi, NFC, infrared, or any other wireless communication protocol) receiving and transmitting components.

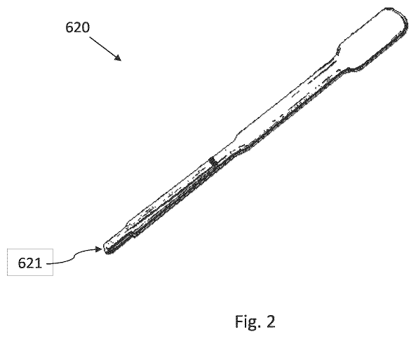

FIG. 2 illustrates a variation of a micropipette 620 that may be used to deliver drops of the liquid sample from the pipette port 621 into or through the inlet port 611.

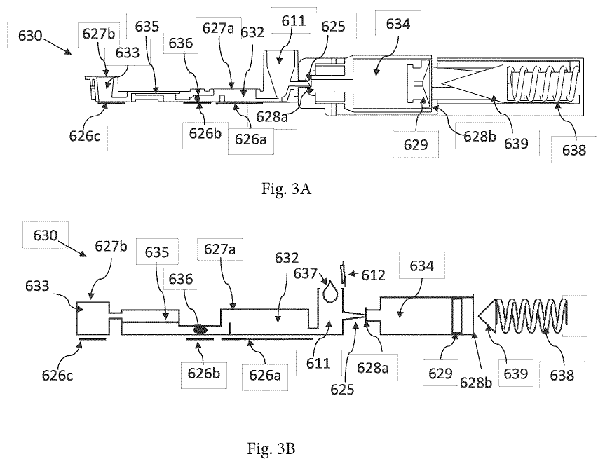

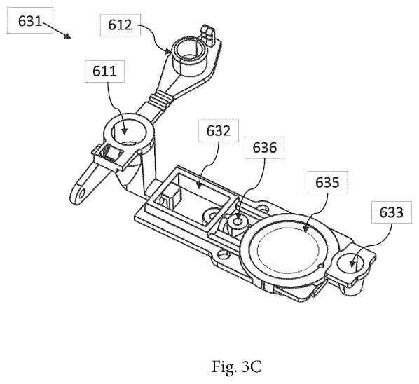

FIGS. 3A through 3C illustrate that inside of the case the device may have a fluidic structure 630, for example, for the sample to flow through during separation and/or analysis. The device may have an actuation structure, for example, to trigger and actuate the device to perform the separation and/or analysis of the sample. The actuation structure may have a spring-loaded syringe. The actuation structure may be actuated by closing the cap 612. The partial fluidic structure 631 includes the inlet port, the process chamber, the filter and the detection chamber,

The device may have an actuator 639. The device may have an actuator spring 638, for example a coil spring, that may be compressed before actuation of the device (e.g., before the cap 612 is closed). The actuator 639 may be in contact with and/or attached to the actuator spring 638. The actuator 639 and/or actuator spring 638 may be in a chamber and/or on a guide that may limit the motion to linear translation in one dimension, for example, parallel with a longitudinal axis of the device.

The device may have a reagent chamber 634 that may be partially or completely filled with flowable reagents (referred to herein as “liquid reagents” and “diluent,” but the reagents may be reactive and/or dilutive and have liquids, solids, such as powders, and/or gasses) before actuation of the device. The liquid reagents may include magnesium, betaine, or combinations thereof.

Anti-coagulants may be added to the inlet port 611 of the device or to the applicator or external collection vial or any other part of the device or system to prevent the blood from coagulating and thereby impede the function of the device. The anti-coagulant can be dried down into the device, coated onto walls, a powder or provided as a lyophilized material in the device. Anti-coagulants that may be considered for this are one or a combination of Sodium Heparin, Lithium Heparin, Dipotassium EDTA, Tripotassium EDTA, Sodium citrate, ACD (Sodium citrate, citric acid, dextrose, potassium sorbate), CTDA (sodium citrate, theophylline, dipyridamole, adenosine), Fluoride/Oxalate, Fluoride/EDTA or any other material that prevents the blood from coagulating.

The reagent chamber 634 may be adjacent to the actuator 639. The reagent chamber 634 may have a plunger 629 that may span the height of the reagent chamber and may have a fluid tight seal against the internal wall of the reagent chamber. The plunger may be slidable within the reagent chamber. Before actuation of the device, the plunger may be adjacent to the actuator 639. The plunger may be separated from the actuator by an actuator seal 628b. The actuator 639 may have a sharp tip that may be configured to pierce the actuator seal 628b.

The liquid reagents may be released by using a spring force that gets released by the closing of the inlet cap 612. Instead of mechanical spring force the system may use compressed gas. The liquid reagents may be released by a manual actuation by the user of the device. The liquid reagents may be released by an actuator.

The reagent chamber may have a pressure sensitive valve and/or reagent chamber seal 628a on an exit channel opposite to the plunger. When the actuator spring is in an expanded configuration, the actuator may press on the liquid reagents, for example increasing the pressure in the reagent chamber 634 enough to open the valve or rupture the reagent chamber seal 628a. The plunger may translate partially or completely across the reagent chamber, pushing some or all of the reagents out of the reagent chamber through the valve or ruptured reagent chamber seal. The reagent chamber may have a valve between the reagent chamber and the inlet port 611, the valve can be any type of valve. The reagent chamber seal 628a may be ruptured by a reagent chamber piercing port 625 in fluid connection with the inlet port 611.

The inlet port 611 may be partially or completely conical with the tip of the cone pointing down. When the cap 612 is open, the sample 637 may be dropped into the inlet port 611, for example from the micropipette 620.

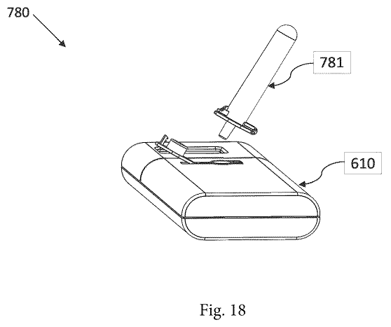

FIG. 18 shows an exemplary embodiment of the system 780 where device 610 can use an external collection vial 781 that may contain chemical components necessary for the separation process, the external collection vial 781 may contain liquid reagents. The external collection vial may receive a swab or a blood sample or other sample types.

The sample may be introduced to reagents in a separate device, for example a blood collection applicator such as a capillary tube or a pipette or an external collection vial 781 or an absorbent pad can be coated or soaked with one or more of the blood separation reagents.

When the reagent chamber seal 628a ruptures and/or the valve on the reagent chamber is open, the reagent chamber 634 may be in fluid communication with the inlet port 611.

The inlet port and reagent chamber can be combined into one chamber that holds liquid and/or dried reagents wherein the sample is introduced by the user.

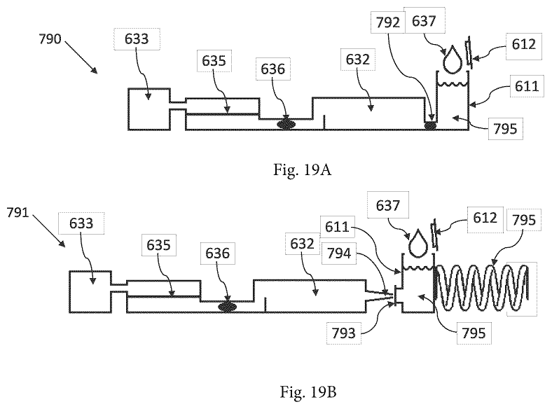

FIG. 19A shows an implementation of the device 790 where the inlet port 611 holds the diluents or liquid reagents 795 and where there is a valve 792 that gets activated once the cap is closed.

FIG. 19B shows an implementation of the device 791 where the inlet port 611 holds the diluents or liquid reagents 795 and where the liquid in the inlet port gets pushed into the reaction chamber once the lid is closed by use of a seal 793 that may be ruptured by a piercing port 794 that may be pierced by force from a spring 795 or by other means.

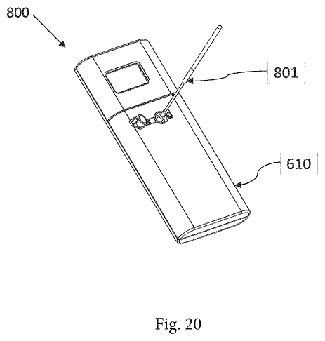

The inlet port 611 may be configured such that a swab may be inserted directly into the inlet port and optionally left in the inlet port during processing. Some swabs are made to be broken off which leaves the possibility to close the cap with the swab inside the inlet port. FIG. 20 shows an embodiment of the system 800 where a swab 801 is inserted into the inlet port of the device 610; in one embodiment the swab is removed before closing the lid, in another embodiment the swab handle is broken off and the swab is left in the device when the lid is closed.

The inlet port 611 may be in fluid communication with a process chamber 632. When the cap 612 is closed, the actuator 639 may be triggered or actuated, pushing the liquid reagents into and through the inlet port 611 mixing the liquid reagents with the sample 637 and pressing the mixed liquid reagents and sample into the process chamber 632. The process chamber 632 may include a gas-permeable process chamber membrane 627a to allow trapped air to escape while liquid is moved into process chamber 632.

The device may have a process chamber heater 626a in or adjacent to (e.g., in the wall of or in contact with the wall of) the process chamber 632. The process chamber heater 626a May for example, heat the mixed sample and liquid reagents to 70° C. to 120° C. for 10 seconds to 20 minutes, more narrowly for 5-10 minutes at 80° C. to 99° C., or even more narrowly for 5-10 minutes at 85-95° C.

The heaters may be any type of technology that generates heat. Examples are resistive heaters, for example one or more conductive heating traces or heating wires adjacent to the area that needs to be heated or conductive sheets or pads or resistors. Part of the fluidic structure can be made of conductive material that may heat when exposed to electrical current. The heaters may be hot fluid that flows adjacent to the areas that need to be heated such as hot air or hot liquid. The heaters may use joule heating of the actual fluid in the device by applying electrical current through the fluid by direct contact or by capacitive contact. The heaters may be infrared heaters. The heaters may be one or more of a semiconductor device such as a fet transistor bjt transistor or diode or other semiconductor device where the heating is generated by the power dissipation in such a semiconductor device. The heat may be generated by a chemical reaction, either by the fluid in the device reacting with reagents or heat may be generated by material external to the fluid in the device creating an exothermic reaction. The heaters may be wireless heaters that dissipate heat directly into the fluid in the device or into some other material in the device or adjacent to the device. The heaters may be any combination of heating technologies.

The process chamber may have an actuatable process chamber exit valve 636 or seal (collectively referred to herein as a process chamber exit valve). The process chamber exit valve may be an electromechanically actuated valve such as a solenoid valve, a thermostat connected to a mechanical valve configured to open at a preselected temperature (e.g., about 92° C. to about 98° C., for example 95° C.), an osmotic pump, a wax valve, an actuatable plastic valve, a low melt metal valve, a membrane or a paper barrier or a chemical valve such as a soluble sugar, or any other barrier that may allow a fluid connection once a preselected temperature or time is reached. The device may have a process chamber valve heater 626b adjacent to the process chamber valve 636. The process chamber heater may be configured to heat the process chamber valve to open the process chamber valve (e.g., melt a wax valve or membrane), for example for about 5 minutes. In some embodiments, a separate process chamber valve heater may be used to open the valve.

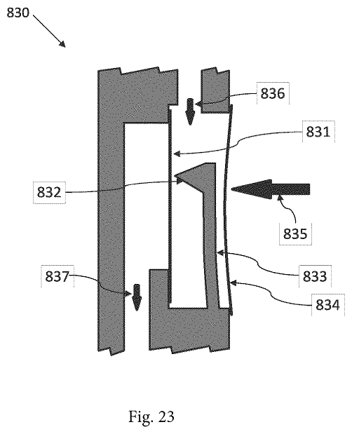

The valve may be a wax valve where the valve blocks the flow of fluid until heat is applied to or near the wax which causes it to melt or break and thereby opening a passage for fluid. Instead of wax it may be any other type of material that melts at a temperature below the melting temperature of other material surrounding the valve; such material may be low temperature metals such as fields metal or it may be other polymers like polycaprolactone or polyethylene. The valve may be a pinch valve. The valve may use electroosmosis; for example the valve may be an electroosmotic pump. As shown in FIG. 23, the valve 830 may include a pierceable membrane 831 that is activated by a force, the force can be external to the fluidic structure by using a flexible barrier that where a force 835 can be translated from the outside of the fluidic structure through a flexible cover 834 to a needle 832 that is attached to a flexible arm 833 pierces a pierceable membrane 831. After the membrane is pierced liquid can flow from the valve inlet 836 to the valve outlet 837. A valve can be activated by heat from a heater or by a mechanical force from a solenoid or a motor or by manual means from a user or by a bimetallic actuator or by shapeshifting from for example nitinol or by any other means or actuators. A heat sensitive process chamber valve (e.g., a wax valve or membrane) may be used with no process chamber valve heater. For example, the heat of the mixed sample and liquid reagents may open the heat sensitive process chamber valve (e.g., melt or break the wax valve or membrane) when the correct temperature is reached by the mixed sample and liquid reagents in the process chamber 632.

The mixed sample and liquid reagents may be under pressure when the process chamber valve opens. For example, the actuator spring pressing via the actuator on the plunger may still be in a partially compressed position, and the plunger may still be free (i.e., unobstructed) to travel toward the reagent chamber seal in the reagent chamber.

The device may have a detection chamber 633 and a processed sample filter 635. The processed sample filter may be between the process chamber exit valve and the detection chamber. When the process chamber valve is opened, the mixed liquid reagents and sample May flow to and be pressed into the processed sample filter. The processed sample filter may filter out inhibitors and/or other non-nucleic acid components of blood. The detection chamber 633 may have a detection chamber heater 626c in or adjacent to the detection chamber. The detection chamber may contain dried or lyophilized reagents required to perform an nucleic acid amplification reaction. The detection chamber 633 may have a gas permeable membrane 627c covering part of the detection chamber to evacuate air or gas when filling the detection chamber.

The valve may be a wax valve where the valve blocks the flow of fluid until heat is applied to or near the wax which causes it to melt or break and thereby opening a passage for fluid. Instead of wax it may be any other type of material that melts at a temperature below the melting temperature of other material surrounding the valve. Such material may be low temperature metals such as fields metal or it may be other polymers like polycaprolactone or polyethylene. If the liquid reagents are released in a specific volume then a valve may not be needed. For example if 200 ul of liquid reagents are released initially then that may only fill up the processing chamber; after the processing chamber heating step then another amount of liquid is released that pushes the processed liquid through the filter and into the detection chamber. In some embodiments, after the first liquid reagent release fills up the processing chamber and the process chamber heating step is complete a separate liquid release or gas release is introduced in the device which pushes the liquid through the filter and into the detection chamber. In some embodiments, after the first liquid reagent release fills up the processing chamber and the process chamber heating step is complete a separate actuator pushes on a flexible or moveable member in the device which pushes the liquid through the filter and into the detection chamber. The actuations can be automatic or manual.

The liquid actions such as valving and fluid movement can be performed using Electro Wetting On Dielectric or Digital microfluidic techniques.

The device may have a centrifuge. The mixed sample and liquid reagents may be centrifuged to separate the components after exiting the process chamber and before the resulting liquid is delivered to the processed sample filter and/or directly to the detection chamber.

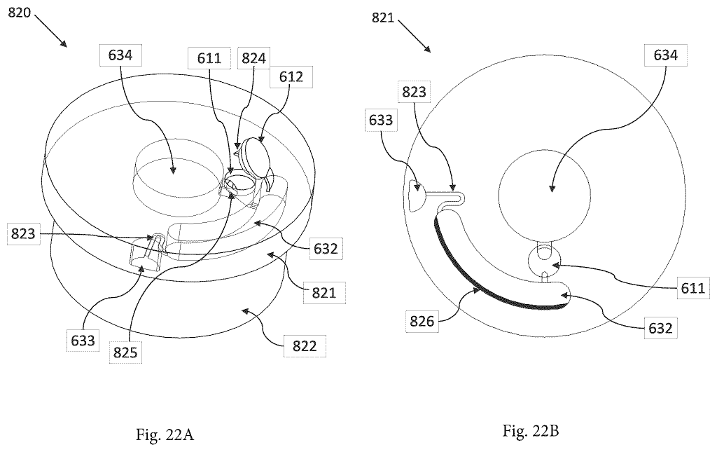

FIGS. 22A and 22B show an exemplary embodiment of a device 820 that uses centrifugal and capillary force for centrifuging processed sample and for fluid movement. Instead of or in addition to using a filter to remove inhibitors from the fluid, the fluid may be centrifuged and the supernatant may be further processed in the detection chamber. In this exemplary device the inlet port 611 the reagent chamber 634, the process chamber 632 and the detection chamber 633 are located on a disk like structure 821 where a motor 822 internal or external to the device supplies the rotating actuation for centrifugation. The rotating action may be supplied by a user for example a user may pull on a string or push a button that translates into rotating all or part of the fluidic structure. When the lid is closed a spike 824 ruptures a pierceable seal 825 which opens a fluid path from the inlet port 611 to the liquid reagent chamber 634 in the center of the disk; the fluid will move from the liquid reagent chamber in the center of the disk to the inlet port 611 where the reagents and blood will move further to the processing chamber 632 upon the centrifugal force from rotation of the disk. The sample and reagent mixture in the processing chamber may be passively or actively mixed by movement of the disk and heated, thereafter the disk will spin so as to centrifuge and separate the dense sample components 826, for example unwanted blood components; after the centrifugation the exit capillary channel 823 from the processing chamber will be filled when the disk is left in a no rotation or slow rotation state; thereafter the disk will spin in order to siphon the upper supernatant in the processing chamber into the detection chamber for amplification and detection. The device may have a detection chamber heater 626c in or adjacent to (e.g., in the wall of or in contact with the wall of) the detection chamber 633.

The detection chamber may be covered with a detection chamber membrane 627b which may be gas permeable.

Gas-permeable membrane is used to vent and degas the fluid in the device and can be added to the device via heat sealing, ultrasonic welding, adhesive, insert molding, or any other method of adding a material to an injection molded part. This membrane can be made from one or a combination of the following materials; PTFE, polypropylene, nylon, polyethersulfone, polyvinylidene fluoride, teflon, polycarbonate, polyethylene, low-density polyethylene, or any other material that can vent air and degas a fluid. The pore size for this venting membrane can range from 0.02 microns to 5.0 microns. This membrane can have hydrophobic and/or hydro-oleophobic properties. The flow rate to vent air out can range from 0.1 slmp (standard liter per minute) to more than 2 slmp.

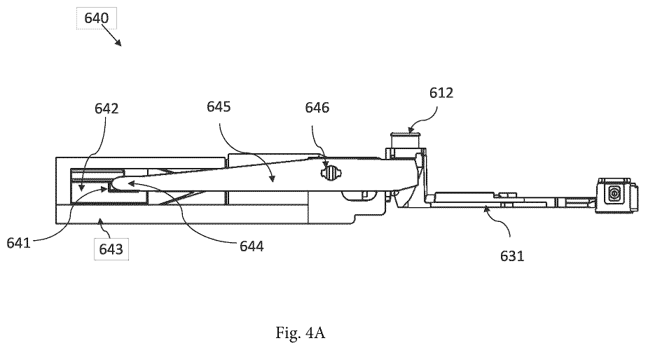

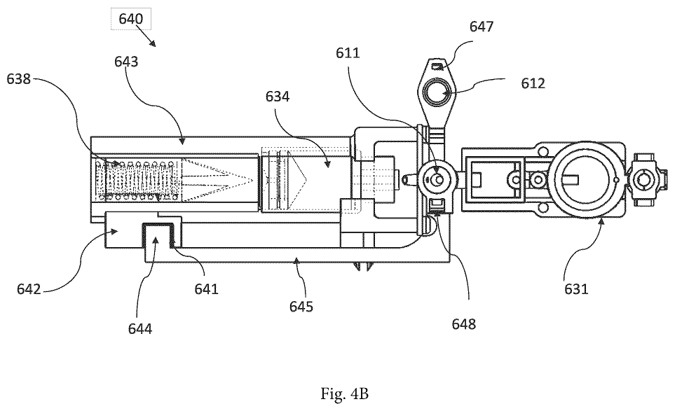

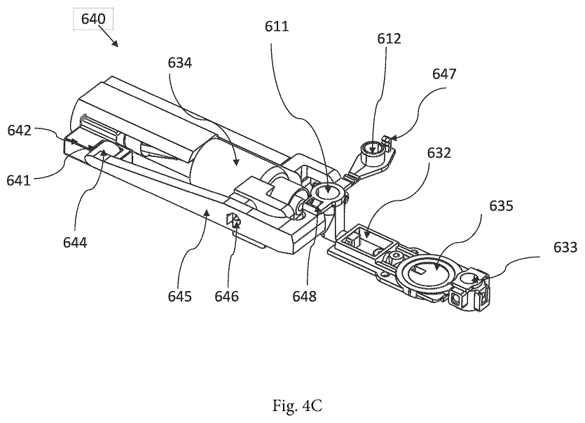

FIGS. 4A through 4C show an exemplary device 640 with an actuating arm 645. The actuating arm may rotate around an actuating pivot 646. The actuating arm may have an arm tab 644 that may extend radially inward from the remainder of the actuating arm at the end of the arm toward the actuator.

The actuator may have an actuator tab 642 extending laterally from the longitudinal axis and/or direction of travel of the actuator. The actuator tab may have an actuator slot 641. The actuator slot may be shaped and sized to receive an interference fit with the arm tab.

When the cap is open and the cap closure tab 647 has not been received by the cap closure port 648 the actuating arm may be in locked configuration. The actuator spring 638 may be fully or partially compressed and the arm tab may be in the actuator slot, for example, interference fitting and preventing translation of the actuator.

The reagent chamber 634 may be encased in a reagent chamber housing 643 and contain liquid reagents for the sample. In some embodiments the device may have a separate reagent chamber that may contain liquid reagents.

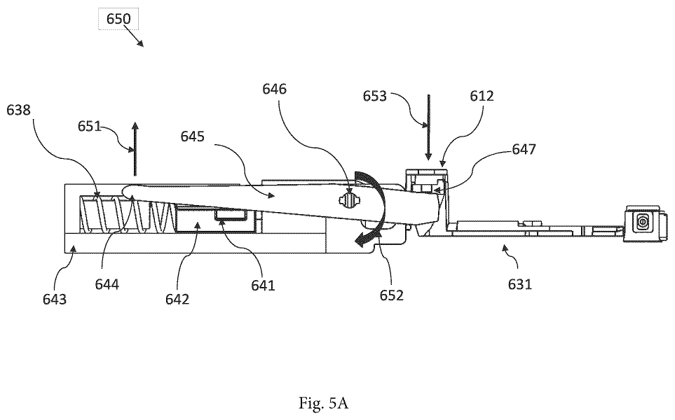

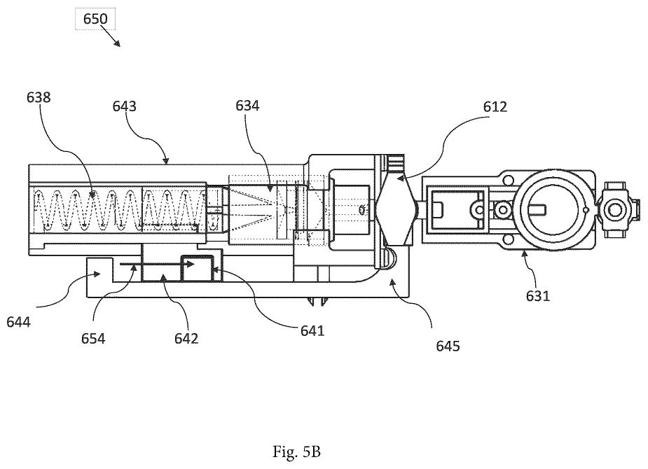

FIGS. 5A and 5B illustrate an embodiment of the device where the actuator may move to an unlocked configuration when the cap is closed 650. When the cap closure tab 647 is inserted into the cap closure port 648 translation of the cap closure tab, as shown by arrow 653, may push down an end of the actuating arm closer to the fluidic structure. The actuating arm may rotate, as shown by arrow 652, around the actuating pivot. The end of the actuating arm closer with the arm tab may translate up, as shown by arrow 651, moving the arm tab out of the actuator slot, freeing the actuator to slidably translate, as shown by arrow 654, pressing the plunger into the liquid reagent, opening the reagent chamber exit seal, and forcing the liquid reagent into the inlet port, and thereby the mixed sample and liquid reagent into and through the fluidic structure.

The translation of the cap closure tab and/or rotation of the actuating arm may close a switch and or press a button activating the heaters and detection chamber components (e.g., sensor and LEDs). The liquid may be sensed with a capacitive sensor or with a liquid sensor or by optical means or by other methods that initiate the heating and further processing steps directly or indirectly via a microprocessor.

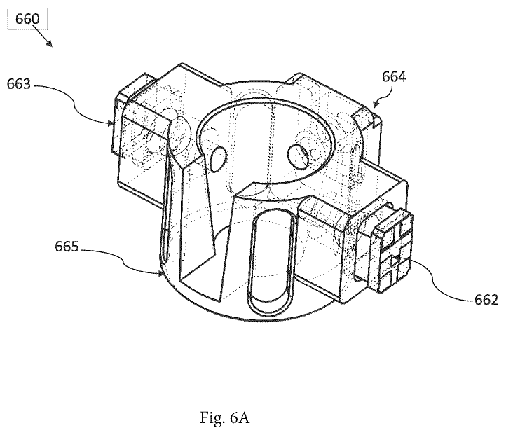

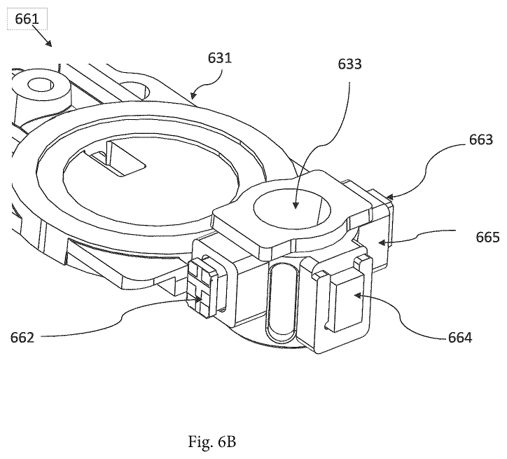

FIGS. 6A and 6B illustrate an embodiment of the device that may have one or more optical sensors. FIG. 6A is an end view of the detection system 660 showing the optical sensor 664, the first LED 662 and the second LED 663 in a metal sleeve 665. a variation of the detection chamber and adjacent elements of the device. FIG. 6B is a close-up view of a portion of a variation of the fluidic system assembly 661 showing the detection chamber 633 with the detection system 660.

In some embodiments, the detection chamber may have a hole or a feature in its wall in which the optical sensor is located.

The device may have the first LED 662 and the second LED 663 on opposite sides of the detection chamber. The LEDs may be positioned 90° away from the optical sensor with respect 6 to the detection chamber, as seen from above. The detection chamber may entirely or partially (e.g., in three locations adjacent to the LEDs and optical sensor) be translucent or transparent. In other embodiments, the optical sensor may be placed on adjacent or opposite, or the same side of the detection chamber as the LED(s). The detection chamber 633 may be located in- or adjacent to a heat conducting sleeve 665 like the metal sleeve or a thermally conductive polymer sleeve for distributing heat around the detection chamber. The sleeve may hold the optical detector 664, LED 662 and LED 663.

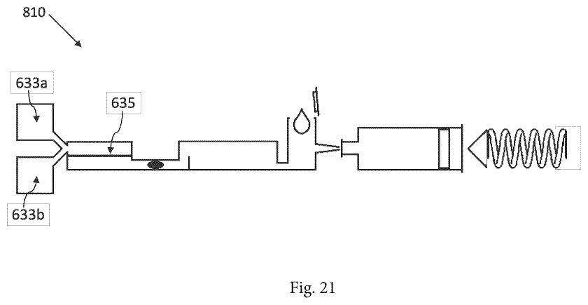

FIG. 21 shows an embodiment of the device 810 where the device may contain more than one detection chamber such as a first detection chamber 633a and a second detection chamber 633b such that the liquid gets split into 1, 2 or more detection chambers after the filter 635, this can increase the number of test targets that can be detected by a device and/or it can provide confirmation of one or more results if it or they are replicated in more than one detection chamber. In another embodiment the device may not contain a detection chamber, instead the processed sample is made available to be extracted from the device for further processing or detection outside the device.

In some embodiments, the device may have one or more digital processors or microprocessors and memory chips, for example on a circuit board. The optical sensor may be in data communication with the microprocessor and/or onboard memory, and (possibly through the microprocessor and/or memory) with the display and/or communication port. The data results from the optical sensor after the sample has been processed may be analyzed and/or digitally processed by the microprocessor in the device, displayed on the display and/or transmitted through the communication port and/or other wireless transmitters in the device to another device, such as smartphone, computer, or any other device than may receive wireless transmission.

An application on a smart phone or on a computer or any other computational device may interface to the device and thereby receive data generated by the device. The application may collect other data such as operator identification, location, altitude, humidity, sound level, sound recording, date, time, temperature, sample identification. If the sample is generated from an animal or human then the application may collect the identification of such animal or human. The application may acquire an image of the animal or human and/or location of the sample acquisition. The data from the device and other data may be stored on the smartphone or computer or any other computational device and/or it may be further transmitted to another database like a cloud storage solution. The application may also transmit data or instructions to the device for guiding processing steps. The application may calculate the final result of a test. The application may function in such a manner that the result of a test from a device may not be readily available to an operator of the test but may transmit the data to another database or computing system and only after approval by another entity is the result of the test made available to the user. The application may function in such a manner that the result of a test from a device may not be readily available to an operator of the test until after a certain time has passed. The application may function in such a manner that the result of a test from a device May not be readily available to an operator of the test until the application is detecting a change in location. The application may acquire other data to add to the stored data associated with a certain test such as weather conditions, location features, situational features such as traffic or lighting conditions or pollution or infection level or any other data that can be collected from other databases or web sites.

An application on a smart phone or on a computer or any other computational device that may interface to the device may initiate the start of the processing of a sample and/or the application may initiate the end of the processing of a sample after a predetermined time or if it determines that enough data has been received to determine a result or if an error has been detected.

The software in the device may end the processing of a sample after a predetermined time or if it determines that enough data has been received to determine a result or if an error has been detected.

The sample process chamber and/or the detection chamber may have additional dried and/or liquid reagents that may be the same or different from the liquid reagents in from the reagent chamber. In an embodiment the device does not contain any liquid reagents but only dry reagents that mixes with the sample before or at the beginning of processing the sample.

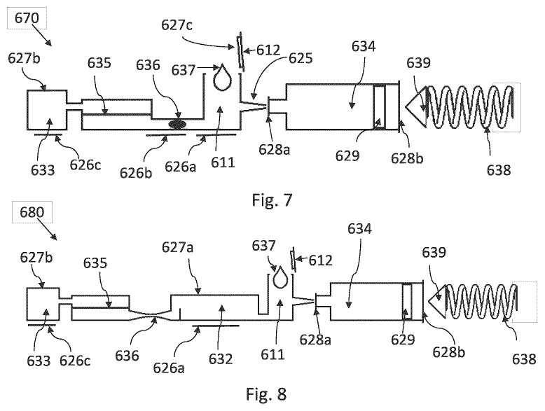

FIG. 7 illustrates an embodiment where the device 670 has no dedicated sample process chamber. Mixing and heating of the sample 637 and the liquid reagents may occur in the inlet port 611. The device may have an inlet port heater that may be configured as the process chamber heater 626a described herein. The device may have an inlet port exit valve that may be configured as the process chamber exit valve 636 described herein. The device may have an inlet port exit valve heater that may be configured as the process chamber exit valve heater 626b described herein. When the inlet port exit valve is open, the inlet port may be in fluid communication with the processed sample filter. The cap 612 may have a cap gas permeable membrane 627c.

FIG. 8 shows an exemplary device 680 that has no process chamber exit valve and/or process chamber exit valve heater. The process chamber may be in fluid communication with the processed sample filter via a channel restriction. The channel restriction may be a narrowing of the conduit between the process chamber and the processed sample filter.

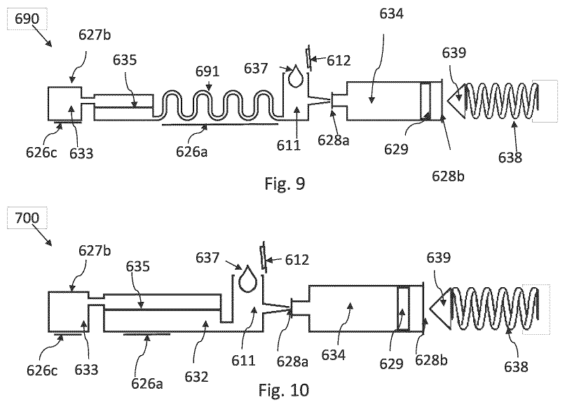

FIG. 9 shows an exemplary device 690 where the cap and/or the detection chamber may have gas permeable membranes. The process chamber may be or have a channel, for example a tube 691. The process chamber heater may be coiled around the tube of the process chamber, integrated into (e.g., in the wall of) the tube, or combinations thereof.

FIG. 10 shows an exemplary device 700 that has no process chamber exit valve and/or process chamber exit valve heater. The process chamber may be in direct fluid communication (e.g., not through the process chamber exit valve) with the processed sample filter. The processed sample filter may be a gas permeable membrane or other membrane that allows for some fluid passage under certain conditions like elevated temperature and/or pressure and/or time and/or fluid type. The detection chamber may be or have a gas permeable membrane. The cap may be or have a gas permeable membrane.

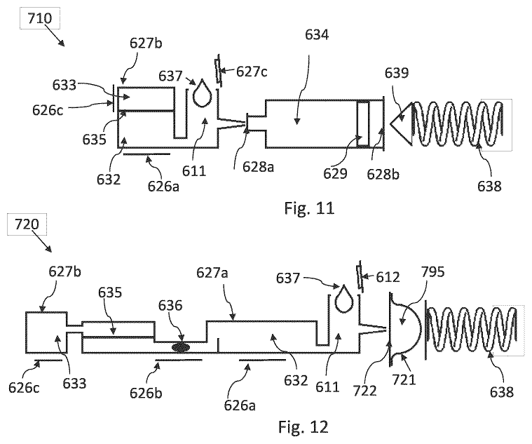

FIG. 11 shows an exemplary device 710 that has no process chamber exit valve and/or process chamber exit valve heater. The sample process chamber may be conjoined with the detection chamber. A single master chamber may be split by the processed sample filter between the sample process chamber and the detection chamber. The master chamber may have the detection chamber heater on the detection chamber side of the master chamber. The process chamber heater may be on a different side of the master chamber from the detection chamber heater.

FIG. 12 illustrates an embodiment 720 where the reagent chamber can have a blister pack, 721. The reagent chamber may be in a chamber or on a track and may be translatable within the case of the device. The actuator may be a flat panel attached to or in contact with the end of the actuator spring. The reagent chamber seal may be a rupturable membrane 722 (e.g., foil) on the blister pack. When actuated, the actuator may push on and translate the reagent chamber into a piercing nozzle. The piercing nozzle may be in fluid communication with the inlet port. The piercing nozzle may puncture the reagent chamber seal when the reagent chamber is translated into the piercing nozzle. The blister pack may be fixed to the fluidic structure and the rupturable seal 722 may be ruptured by pushing on another side of the blister pack 721 whereby pressure is built up inside the blister pack 721 and where the pressure results in rupturable seal 722 deforming to a point where it engages the piercing nozzle and creates a fluid path from the liquid reagents 795 inside of the foil pack to the inlet port 611.

The actuator-side of the reagent chamber may be flexible. The blister pack 721 or reagent chamber may be held fixed within the case. The actuator may press and collapse the actuator-side of the reagent chamber, increasing the pressure in the reagent chamber, for example, causing the reagent chamber seal to rupture (e.g., with or without having been pierced by the piercing nozzle).

The amplification reagents (Oligonucleotides, Enzymes, dNTPs and buffer components) may be lyophilized or dried down inside the fluidic structure so that they may last for an extended period of time at room temperature—and then when the reaction is going to happen the dried or lyophilized reagents will be reconstituted with the liquid reagents. In order for some reagents to maintain viability for an extended time they are dried down or lyophilized in the device where they will maintain viability as long as they are kept dry, therefore the liquid reagents needs to be separated from the dried or lyophilized reagents, this is done by containing the liquid reagents in low liquid permeability material such as metal foil and/or plastic or other materials. The whole device may be packaged inside a metal foil pouch that also contains desiccant to absorb any liquid that may migrate from the outside of the metal foil pouch or from the liquid reagent chamber.

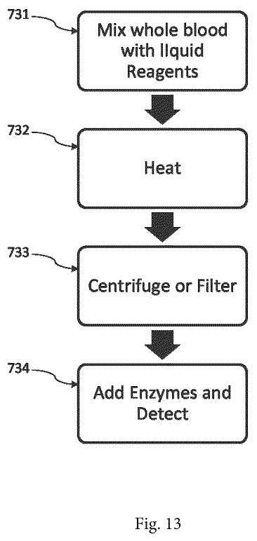

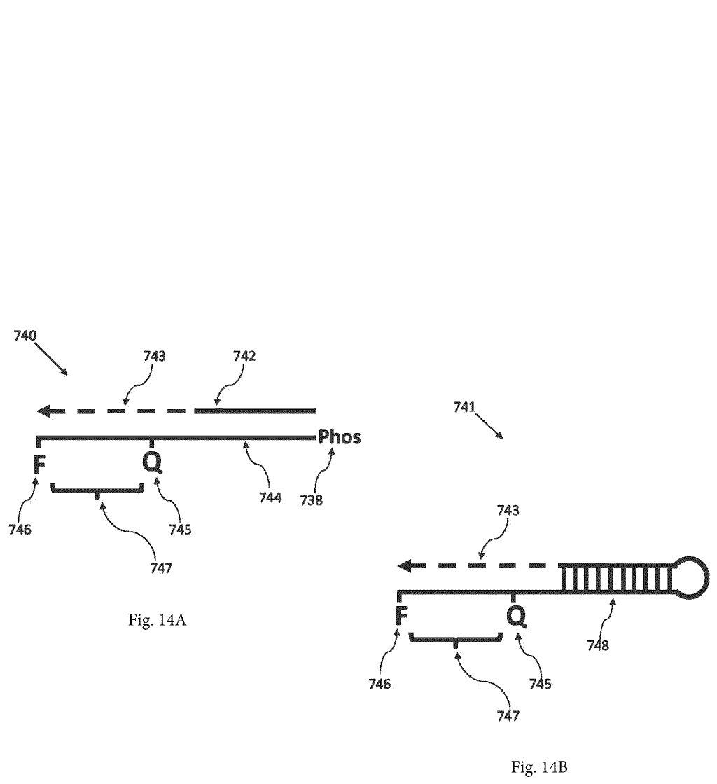

FIG. 13 shows a typical assay flow for a whole blood sample. The whole blood sample may be mixed with the liquid reagents 731 containing magnesium ions plus all other thermostable amplification reaction reagents. The liquid reagents may contain Magnesium Sulfate from 3 mM to 5 mM, Tris buffer at pH 8.8, 0.5M Betaine, and oligonucleotides. The sample may be heated 732 to preferably above 85 degrees celsius for 5 minutes or longer, the sample may then be filtered or centrifuged 733 to separate the precipitate. The remaining supernatant may be combined with lyophilized or dried down enzymes or other reaction components. A nucleic acid amplification and/or detection event may then occur 734.

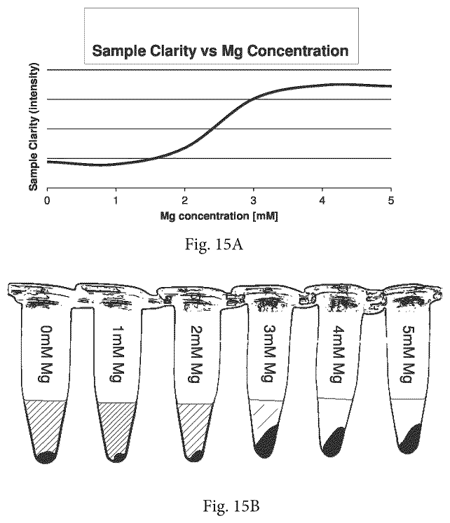

FIG. 14 shows possible control oligo configurations, these designs may control for reaction viability without having to perform a full control amplification reaction which can take up precious reaction resources when multiplexing. FIG. 14A shows design 740 containing a control oligonucleotide that has a region 744 that is complementary to a complementary oligonucleotide 742, a region that contains a restriction enzyme recognition sequence 747 separating a fluorophore 746 and a fluorescent quencher 745. This design may contain a 3′ extension blocker 738. In the presence of viable nucleic acid amplification reactants and reaction conditions, the complementary oligonucleotide may be extended in the region 743, making a double stranded region containing the restriction enzyme recognition sequence that may be cleaved by a restriction enzyme, resulting in increased fluorescence from the fluorophore. FIG. 14B shows a design of a control oligonucleotide 741 containing a self-complementary loop structure 748, a region that contains a restriction enzyme recognition sequence 747 separating a fluorophore 746 and a fluorescent quencher 745. In the presence of viable nucleic acid amplification reactants and reaction conditions, the three prime end of the control oligonucleotide may be extended in the region 743 making a double stranded region containing the restriction enzyme recognition sequence that may be cleaved by a restriction enzyme, resulting in increased fluorescence from the fluorophore.

FIGS. 15A and 15B show the dependance of the precipitate formation on the magnesium concentration in the heated mixed liquid sample and liquid reagents. The magnesium concentration was varied from 1 mM to 5 mM in the liquid reagent which also comprised 20 mM Tris buffer, pH 8.8, and 0.5M betaine. After mixing the liquid blood sample with the liquid reagents, the solutions were heated for 5 minutes at 95 degrees Celsius and then centrifuged at 7000 rpm for 3 minutes. The clarity of the resultant supernatant was analyzed using image J (NIH) and graphed in FIG. 15A and shown in FIG. 15B.

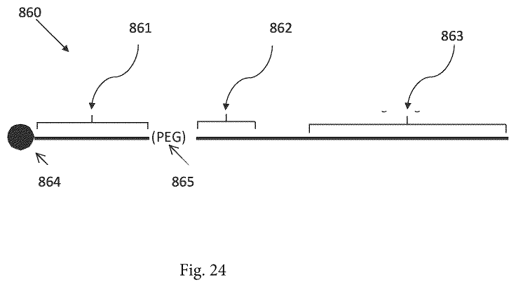

In some embodiments, the detection reagent may include a reporter oligonucleotide. FIG. 24 shows an example of the reporter oligonucleotide design 860. The reporter oligonucleotide contains a portion of the reporter oligonucleotide 863 on 3′ end that is complementary to the desired nucleic acid target in a region internal to the amplicon primer sites, a restriction enzyme recognition site 862, a polymerase extension blocker site 865 and a portion of the reporter oligonucleotide that contains a zip code region 861 with an optional detectable tag 864. During the course of the reaction, the probe may become double stranded in the region that is on 3′ side of the extension blocker. This initiates strand cleavage at the recognition site and releases the single stranded zip code region. The zip code region may contain a detectable tag that allows for the zip code region to be detected at a subsequent hybridization-based event such as hybridization to a non-target specific oligonucleotide capture probe. The oligonucleotide capture probe may be in solution. The oligonucleotide capture probe may be on a surface. The surface may be flat or spherical. The surface may be a bead. The surface oligonucleotide capture probe may be part of an array of oligonucleotide capture probes. The surface may be an electrode in an electrochemical cell. This tag may be an electrochemical molecule, such as ferrocene or methylene blue, that will participate in electron transfer with a solid phase electrode or a fluorescent tag that will fluoresce when excited by the appropriate wavelength of light. The tag could also be a molecule exhibiting electrochemiluminescent properties. The reporter oligonucleotide may contain secondary structure preventing portion of the reporter oligonucleotide from hybridizing to a non-target specific oligonucleotide capture probe. During the course of the amplification reaction, a portion of a reporter oligonucleotide may gain the ability to hybridize to a non-target specific oligonucleotide capture probe as a result of the presence of the target

In many applications it is desirable to be able to perform multiplexed genetic analysis in a low cost, low power, fast manner. This reporter oligonucleotide may be incorporated into a strand-displacement, isothermal nucleic acid amplification assay to indicate the presence of a nucleic acid target under these types of requirements. The isothermal amplification may be detected when the cleaved zip code region of the reporter oligonucleotide, containing an electroactive tag, hybridizes to oligonucleotide capture probes covalently bound to an electrode or array of electrodes in an electrochemical cell. When an appropriate potential is applied to the electrode vs. a reference electrode, electrons are transferred between the electroactive tag and the electrode surface to produce a detectable current.

The system may allow for detecting multiple nucleic acid sequences in a chamber by having the result of individual sequences hybridize to specific locations on a surface in the container, where the individual sequence strands or representations of the strands have a fluorescent tag that is measured using fluorescent lifetime. In other embodiments, the system for detecting multiple nucleic acid sequences in a chamber by having the result of individual sequences hybridize to one or more beads in the container, where the individual sequence strands or representations of the strands have a fluorescent tag that is measured using fluorescent lifetime. In some embodiments, the system for detecting fluorescence in a chamber by a fluorescent tag that is measured using fluorescent lifetime.

Detection methods may include optical fluorescent detection, Light absorbance, Light transmittance, Optical reflectance, Electrochemical, Electrical, Resistance and any other methods for detecting analytical assay reactions.

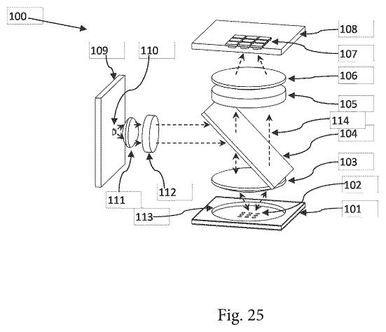

In some applications it is preferable to use fluorescent detection. In this case, the tag on the cleaved zip code region is a fluorescent molecule that will fluoresce when excited at the appropriate wavelength after hybridization to surface capture probes or an array of surface capture probes. FIG. 25 shows an epifluorescent detection setup 100 where fluorescent spots are being measured by using conventional fluorescent measurement where the emission light wavelength is separated from the excitation light wavelength by use of optical filters. The setup 100 can have a member 101 with a sample chamber 113 wherein fluorescent spots 102 are located; the sample chamber 113 has a transparent lid where light may pass through to and from the fluorescent spots 102 the sample chamber may also be made with no lid and the light will pass through the surface of the liquid or the optical sensor may detect the spots through a transparent bottom or a transparent side of the chamber 113. The fluorescence is to be measured of the various spots by exposing them to light of a certain wavelength and then measuring how much light is emitted at another wavelength. This may for example be used to measure various hybridized nucleic acid probes in order to identify specific nucleic acid sequences. The emitted light 114 passes through a lens 103 which collimates the light before passing through a beam splitter 104 and a detection filter 105 which filters out the excitation light from light source 110 and then through a detection lens 106 which focuses the light onto an array of detectors 107 on board 108. The excitation light comes from a light source 110 which could be a light emitting diode or a laser diode placed on board 109 or other type of light source with appropriate wavelength. The excitation light then passes through the excitation lens 111 which collimates the light before passing through excitation filter 112 onto beam splitter 104 and further through lens 103 onto the spots 102. The lenses 103, 111 and 106 helps collimate the light that passes through the optical filters 112 and 105 so that the filters work optimally, which is the case with most optical filters particularly optical interference filters. The beam splitter 104 may also work better with collimated light, particularly if the beam splitter 104 is a dichroic interference filter.

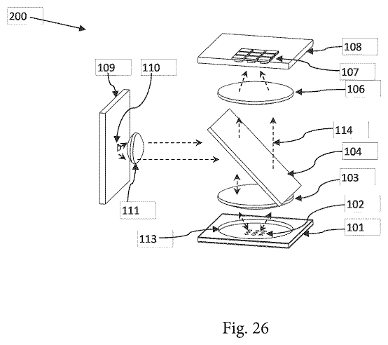

A more compact, lower cost fluorescent detection module may be achieved using time-resolved fluorescent measurements. The label on the cleaved zip code region can be a fluorescent molecule with a fluorescent lifetime appropriate for time-resolved measurements that is excited at the appropriate wavelength after hybridization to surface capture probes or an array of surface capture probes. Tris-(2,2′-bipyridine) ruthenium is an example of such a fluorophore that may be covalently bound to an oligonucleotide and has a long fluorescent lifetime of several hundred nanoseconds. FIG. 26 shows a detection setup 200 where fluorescent spots 102 are being measured by using fluorescent lifetime where the emission light is separated from the excitation light by time. The setup 200 can have a member 101 with a sample chamber 113 wherein fluorescent spots 102 are located, the sample chamber 113 has a transparent lid where light may pass through to and from the fluorescent spots 102, the sample chamber may also be made with no lid and the light will pass through the surface of the liquid or the optical sensor may detect the spots through a transparent bottom or a transparent side of the chamber 113. The fluorescence is to be measured of the various spots by exposing them to light at a certain point in time and then measuring how much light is emitted at another point in time. This assumes that the spots have fluorophores that have a fluorescent lifetime that is long enough for the excitation and detection components to turn on and off and perform a measurement, such a fluorophore could be Tris-(2,2′-bipyridine) ruthenium with a fluorescent lifetime of several hundred nanoseconds. This may for example be used to measure various hybridized nucleic acid probes in order to identify specific nucleic acid sequences. The emitted light 114 passes through a lens 103 which collimates the light before passing through a beam splitter 104 and a detection lens 106 which focuses the light onto an array of detectors 107 on board 108. The excitation light comes from a light source 110 which could be a light emitting diode or a laser diode placed on board 109 or other type of light source with appropriate wavelength and speed to excite the fluorescent spots 102. The excitation light then passes through the excitation lens 111 which collimates the light before hitting beam splitter 104 and further through lens 103 onto the spots 102. The detection module 200 is simpler than the module 100 by not having the optical filters 105 and 112. This may lead to a more compact and lower cost module. 6

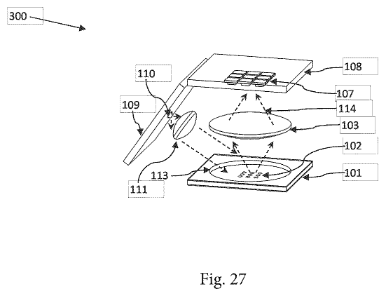

In some applications it is preferable to have an even more compact, lower cost fluorescent detection module. FIG. 27 shows a detection setup 300 where fluorescent spots 102 are being measured by using fluorescent lifetime where the emission light is separated from the excitation light by time. The setup 300 can have a member 101 with a sample chamber 113 wherein fluorescent spots 102 are located, the sample chamber 113 has a transparent lid where light may pass through to and from the fluorescent spots 102, the sample chamber may also be made with no lid and the light will pass through the surface of the liquid or the optical sensor may detect the spots through a transparent bottom or a transparent side of the chamber 113. The fluorescence is to be measured of the various spots by exposing them to light at a certain point in time and then measuring how much light is emitted at another point in time. This assumes that the spots have fluorophores that have a fluorescent lifetime that is long enough for the excitation and detection components to turn on and off and perform a measurement, such a fluorophore could be Tris-(2,2′-bipyridine) ruthenium with a fluorescent lifetime of several hundred nanoseconds. This may for example be used to measure various hybridized nucleic acid probes in order to identify specific nucleic acid sequences. The emitted light 114 passes through a lens 103 which focuses it onto an array of detectors 107 on board 108. The excitation light comes from a light source 110 which could be a light emitting diode or a laser diode placed on board 109 or other type of light source with appropriate wavelength and speed to excite the fluorescent spots 102. The excitation light then passes through the excitation lens 111 which focuses the excitation light directly onto the fluorescent spots 102. The module 300 is an even simpler module than the module 100 by not having the optical filters 105 and 112 and therefore not needing the lens 106 as well. This simplification further enables the elimination of the beam splitter 104. This may lead to a very compact and lower cost module. The module may be further simplified in cases where the sensor array 107 may be located so that individual detectors in the sensor array 107 have lenses and therefore lens 103 may be eliminated. The light emitter 110 may potentially project light directly onto the fluorescent spots 102 without a separate lens 111 or the emitter may itself have a lens as part of the component which would be directional. The sensor array 107 may be an array of photodiodes and/or a 2D image sensor such as a CMOS or CCD with a fast shutter function that may blank out the image sensor when the emitter 110 is turned on or the detector array 107 may have a separate shutter. Using fluorescent lifetime May have advantages when it comes to background light since much background light will not have fluorescent lifetime in the same range as the measured fluorophore.

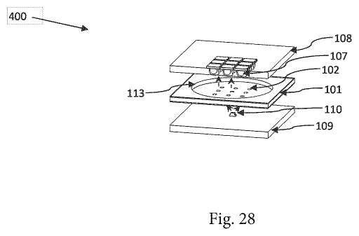

FIG. 28 shows the simplest detection setup 400 where fluorescent spots 102 are being measured by using fluorescent lifetime where the emission light is separated from the excitation light by time. The setup 400 can have a member 101 with a sample chamber 113 wherein fluorescent spots 102 are located on a transparent surface where light may pass from a light source 110 onto the fluorescent spots 102, the sample chamber 113 has a transparent lid where light may pass through from the fluorescent spots 102, the sample chamber may also be made with no lid and the light will pass through the surface of the liquid or the optical sensor may detect the spots through a transparent bottom or a transparent side of the chamber 113, the excitation light and the emission light may be exposed and detected from the same side of the chamber 113 or from opposite sides of the chamber 113. The spots 102 may be on an open surface when being read, the spots may be in surrounding liquid or in a mostly dry surrounding when read. The fluorescence is to be measured of the various spots by exposing them to light at a certain point in time and then measuring how much light is emitted at another point in time. This assumes that the spots have fluorophores that have a fluorescent lifetime that is long enough for the excitation and detection components to turn on and off and perform a measurement, such a fluorophore could be Tris-(2,2′-bipyridine) ruthenium with a fluorescent lifetime of several hundred nanoseconds. This may for example be used to measure various hybridized nucleic acid probes in order to identify specific nucleic acid sequences. The emitted light is captured onto an array of detectors 107 with individual lenses on board 108. The excitation light comes from a light source 110 which could be a light emitting diode or a laser diode placed on board 109 or another type of light source with appropriate wavelength and speed to excite the fluorescent spots 102. The excitation light then passes through the lower part of the chamber 113 onto the fluorescent spots 102. The light source 110 may have no lens or a built-in lens or a separate lens to direct more light onto the fluorescent spots 102. The module 400 is an even simpler module than the module 100 by not having the optical filters 105 and 112 or beam splitter 104 and fewer lenses as well. This may lead to a very compact and lower cost module. The sensor or detector array 107 may be an array of photodiodes or photo transistors, other photo sensitive components, a 2D image sensor such as a CMOS or CCD with a fast shutter function that may blank out the image sensor when the emitter 110 is turned on, have a separate shutter, or combinations thereof. For the sensors 107 to capture light from specific spots they may have lenses on the sensors themselves or they may have a lens array separate from the sensors or they may have a separate common lens between the fluorescent spots and the detectors or they may have a combination of lenses and lens types. Other ways of directing light from fluorescent spots onto specific sensors is by using light guides or by having absorbing or reflective walls separating light from various spots onto specific sensors, in that case no lens may be needed. Fluorescent lifetime detection will also work for measuring a single spot or one or more chambers with or without the need for lenses. Using fluorescent lifetime may have advantages when it comes to background light since much background light will not have fluorescent lifetime in the same range as the measured fluorophore. Other fluorophores may be used that have fluorescent lifetime where detectors and light sources may be turned on and off within the time of there being fluorescence from excitation of the fluorophore, examples are Pyrene and 2-Aminopurine and others. Almost all fluorescent probes have a measurable fluorescent lifetime, but this simplified detection module may use lower cost components if fluorophores may be used with longer fluorescent lifetimes in the order of several nanoseconds.

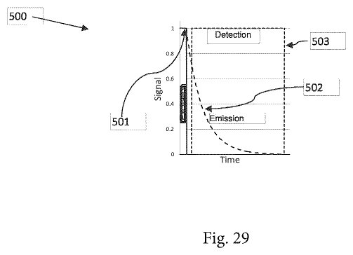

FIG. 29 shows a time diagram 500 of how the fluorescent light may be measured from a fluorophore when using the fluorescent lifetime to distinguish the excitation and emission light. The Emission light is turned on for a short period of time 501 then the emission light starts to emit 502 with a decaying trend. After the excitation light has been turned off then the detectors can start to measure 503 the light 502 before all the emitted light has decayed. After the detector stops to detect then the whole cycle may be repeated starting with turning on the emission light. An alternative or additional step to measuring lifetime by turning on and off the emission light is to modulate the emission light and then detect the modulation lag in the sensor light, for example by using a sine wave modulation or a square wave modulation of the excitation light and measuring the phase difference in the detected light, this may require optical filters in order to separate the emission and detection light. An issue with fluorescent measurement of spots in a liquid surrounding is that the liquid surrounding may contain un-bound fluorophores which will fluoresce. As such a detector or multiple detectors may be used to get a reference measurement of bulk liquid fluorescence and correct readings from spots with the bulk fluorescence reading. Another method is to measure the spots before a reaction or in the beginning of a reaction or throughout a reaction and thereby detect changes in fluorescence as the reaction occurs which would indicate if a spot has a hybridized probe. 6

Oligonucleotides labeled with fluorophores with long fluorescent lifetimes may also be attached to a surface in contact with a solution phase isothermal amplification reaction to act as capture probes that may monitor the course of the amplification reaction in real time. This may be accomplished by labeling the zip code region of the reporter probe with a fluorescent quencher that will quench the fluorescence of the labeled capture probe upon hybridization. This will eliminate fluorescent background in the solution. The fluorescence from the labeled capture probe may be detected using the fluorescent lifetime to distinguish the excitation and emission light.

Oligonucleotides labeled with fluorophores with long fluorescent lifetimes attached to a surface may also be in contact with a solution phase polymerase chain reaction. The solution phase polymerase chain reaction may contain a reporter oligonucleotide probe labeled with a quencher that is complementary to the capture probe on the surface as well as to the amplicon in the solution phase reaction. The quencher-labeled reporter oligo may be complementary to the amplicon in a region internal to the primer sites so that in the presence of a polymerase with nuclease activity, it gets digested or cleaved during the course of the amplification reaction. The fluorescence from the labeled capture probe spot may be detected in real time during the polymerase extension stage of the reaction using the fluorescent lifetime to distinguish the excitation and emission light. As the solution phase reporter oligonucleotide probe gets digested during the course of the amplification, less is available at each cycle to hybridize to the surface and quench the fluorescence of the labeled capture probe. This causes the fluorescence of the labeled capture probe spot on the surface to increase at each cycle if amplification is proceeding.

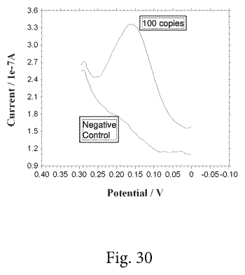

FIG. 30 shows a signal from an electrochemical detection pad where a cleaved zip code region from a reporter oligonucleotide that was a reactant in a nucleic acid amplification reaction in the presence of target has bound to a surface probe, labeled 100 copies, compared to a negative control.

### Methods of Use

In some embodiments, the device may be used to detect African swine fever (ASFV), Equine herpesvirus (EHV), Equine Infectious Anemia virus, Equine influenza, Porcine Epidemic Diarrhea Virus (PEDv), Salmonella, Escherichia coli, Coronaviruses, Chlamydia trachomatis, Neisseria gonorrhoeae, Human Immunodeficiency Virus, Treponema pallidum, and any other viral, bacterial, plant, or mammalian nucleic acid, recombinant DNA, perform PCR, isothermal amplification methods, or combinations thereof. For example, the device may be used in combination with the respective portions of USDA document number SOP-DS-0071, revision 03, release date 9 Aug. 2018, titled “Preparation, Performance, and Interpretation of the African Swine Fever rPCR Assay on the Applied Biosystems 7500 Real-time PCR System,” which is incorporated by reference herein in its entirety.

The device may be packaged in a clean and sterile sealed foil pouch. A micropipette may be packaged in the same pouch or a separate pouch. The user may remove the device and micropipette from the pouch(es).

The device may be placed on a flat surface with the screen facing upwards.

The user may micropipette whole blood, or other nucleic acid sample into the inlet port or hole of the device.

The user may close the cap tightly, hearing an audible click and/or feeling a tangible snap when the cap is sealed and the cap closure tab actuates the device.

In some embodiments, the cap will self-lock and trigger the device to start the test when the cap is completely pressed down and closed.

In some embodiments, after the analysis is complete, the result may be displayed on screen, the screen may indicate that the results are ready, the results may be transmitted to another device, or results may be communicated to the user in another way.

Closing and pushing the cap sealed shut may trigger pushing down on the actuating arm lever that releases the actuator spring from a compression state to puncture and pierce through a foil-sealed barrel (i.e., the reagent chamber) that may have, for example, 1 mL of a diluent solution (i.e., liquid reagents).

The diluent solution (i.e., liquid reagents) may include an isothermal buffer, MgSO4, MgCl2, betaine, nuclease-free water, or combinations thereof.

The diluent solution (i.e., liquid reagents) may travel through a microfluidic chip to mix with the sample and fill the process chamber. The liquid sample may be mixed at a 1:10 dilution ratio with the liquid reagents. The process chamber may have dried oligos that may be reconstituted and mixed by the mixed sample and liquid reagents.

The process chamber may be used for heating. Localized heaters adjacent to the chamber may heat the mixed sample to about 90 degrees Celsius for about 5 mins. This process chamber may have a process chamber exit channel that may be sealed by the process chamber exit valve; the process chamber exit valve may be a wax valve.

Once heating is completed, the wax valve may be actuated by a separate heater (i.e., the process chamber exit valve heater) to melt the wax and allow the sample and liquid reagents from the process chamber to flow through the microfluidic chip and into the next phase.

The wax valve may be made from paraffin wax (e.g., McMaster cat #: 93955K73).

This denatured sample may then be passed through the processed sample filter, for example a track-etched membrane filter, for example, to remove inhibitory components. The processed sample filter may be a 5.0 micron track-etched filter membrane (e.g., Sterlitech part #: PET5025100).

The filtered sample may fill the detection chamber for detection. The detection chamber may have a dried enzyme mix that may be reconstituted and mixed by the filtered sample.

The detection chamber may be heated from an adjacent heater to 60 degrees Celsius and kept constant for the duration of optical measurement.

The detection chamber may be transparent and surrounded by a detection module. The detection module may have two LEDs (e.g., KingBright part #: APDA3020VBC/D and KingBright part #APDA3020SYCK/J3-PF) pointing across the chamber and an optical sensor (e.g., AMS part #: AS7341) viewing perpendicular to the light beam measuring fluorescent count levels. A count change resembling a sigmoid curve may be considered a positive result.

After 20 minutes of optical measurements, the microprocessor on the device may run data through an algorithm to verify if the sample is positive or negative.

The display may show the final result. The display may show instructions for use, the status of the device, results of the separation and/or analysis of a liquid sample. The display may show the results of the analysis of a liquid sample as resultant metrics and/or encoded text or a symbol (e.g., a QR code). The device may delay displaying the results until a code is entered (wirelessly or via a keypad and/or biometric component on the case, not shown) and/or a preset amount of time has passed (e.g., 12 hours) since the completion of the analysis.

The method may be performed entirely using pipettes and existing mixing vessels and heaters without an integrated fluidic device. For example, the following protocol may be used with or without the disclosed devices:

1. Isothermal fluorometer, Agdia Amplifire (part #: AFR 60400), is used for detection for benchtop protocol.

### 2. Preparation of Primer/Probe Mix:

a. 2.5 ul of Primer 1 (IDT);
b. 2.5 ul of Primer 2 (IDT);
c. 2.5 ul of Bumper 1 (IDT);
d. 2.5 ul of Bumper 2 (IDT);
e. 2.5 ul of Probe (BioSearch); and
f. 37.5 ul of Nuclease-free water (IDT product #: Nov. 4, 2002-01).
   3. Preparation of dNTP Mix:
a. 4 ul dCTP (TriLink cat #N-8002-10);
b. 2 ul of dTTP (Thermo cat #: 10297018);
c. 2 ul of dATP (Thermo cat #: 10297018);
d. 2 ul of dGTP (Thermo cat #: 10297018); and
e. 10 ul of Nuclease-free water (IDT product #: Nov. 4, 2002-01).

### 4. Preparation of Reagent 1:

a. 50 ul of Isothermal Buffer (NEB cat #: B0537S);
b. 50 ul 10× Primer/Probe Mix;
c. 50 ul of 5M Betaine (Sigma Aldrich: cat #: B0300-1VL); and
d. 215 ul of Nuclease-free water (IDT product #: Nov. 4, 2002-01).

### 5. Preparation of Reagent 2:

a. 5 ul of MgSO4 (NEB cat #: B1003S); and
b. 20 ul of dNTP mix.

### 6. Preparation of Enzyme Mix

a. 12.5 ul of WarmStart BST 2.0 (NEB cat #: M0538S); and
b. 42.5 ul of BSOBI (NEB cat #: R0586S).
   7. Aliquot 39 ul of Reagent 1 into PCR tube.
   8. Add 3.9 ul of whole blood into the PCR tube with Reagent 1. Mix by pipetting up and down.
   9. Put the PCR tube into a thermocycler for 5 minutes at 95 degrees Celsius.
   10. Centrifuge PCR tube for 3 minutes in microcentrifuge (×7,000 g).
   11. Pipette out 18.25 ul of supernatant of tube and add into a new PCR tube.
   12. Add 1.25 ul of Reagent 2 into the PCR tube. Mix by pipetting. Vortex and centrifuge for 20 seconds.
   13. Load PCR tube into Amplifire instrument, wait until instrument reaches 61 degrees Celsius.
   14. Add 5.5 ul of Enzyme Mix PCR tube and mix by pipetting up and down (6-8 times). Press start.

EXPERIMENT

| | Reference | Disclosed | Disclosed |
|---|---|---|---|
| | rtPCR | method ASFV | method Internal |
| Dilution | Ct value | result | Positive Control |
| undiluted | 21 | + | + |
| 10\hat{ }−1 | 25 | + | + |
| 10\hat{ }−2 | 28 | + | + |
| 10\hat{ }−3 | 31 | + | + |
| 10\hat{ }−4 | 35 | + | + |
| 10\hat{ }−5 | 37 | + | + |
| 10\hat{ }−6 | 40 | + | + |
| 10\hat{ }−7 | Not Determined | + | + |
| Negative | Not Determined | − | + |
| Control | | | |

Whole blood samples were obtained from swine experimentally infected with African Swine Fever and serially diluted with non-infected swine blood. Samples were run using the APHIS protocol with MagMax Kit and compared to protocol disclosed herein and outlined in the bench level protocol. Results show better sensitivity with the protocol disclosed herein.

### Example 2

User removes the device from a sealed foil pouch filled with desiccant packets. The ASFV device contains an E-ink display, instructing the user to “add a sample and close cap”. The device must be placed on a flat surface. The blood sample can be 20 uL of whole blood from a naturally ASFV infected pig. Using the micropipette that is metered for 20 uL, the blood sample is dispensed into the inlet hole of the device. The inlet cap, attached to the outer case of the device, is then closed shut. The closing of the cap triggers the start of the test by both releasing a spring-actuating diluent dispensing and starting the automated sequence of the circuit board. The liquid dispensing occurs by having a plastic actuator driven by a compression spring to pierce through a lidding of a diluent container, containing 700 uL of diluent, and push diluent into the main fluidic chip. The diluent is first pushed through the inlet port where the blood sample was added. The fluid is driven by excess diluent that is stored in the diluent container and spring supplying constant force. The diluent solution and blood sample get mixed and pushed further into the next chamber of the fluidic chip, process chamber. The process chamber is used to heat denature the blood-diluent mixture to 95 Celsius for 5 minutes. This chamber contains a lyophilized bead containing oligos specific for ASFV. A flex circuit board underneath the fluidic chip heats this localized region to the target temperature and duration. This region of the fluidic chip is constructed by a heat-scaled aluminum foil on the bottom and a gas-permeable venting membrane on top to release any air in the fluid path. The process chamber's exit channel is initially closed by a wax valve. After heating the process chamber for 5 minutes, the wax valve is opened by melting the wax, at 65 Celsius, and allowing liquid to be pushed through the channel and into the next chamber. Next chamber contains a 5 micron pore size filter membrane to remove blood precipitate and other assay inhibitors from the processed sample solution. The filter membrane also traps solidified wax from the wax valve. The filtered solution is then pushed into the last chamber of the fluidic chip for fluorescent detection. The detection chamber contains another lyophilized bead which holds the dNTPS and enzymes for the reaction. This chamber is heated to 60 Celsius and held constant for 20 minutes. Exterior to the chamber are two LEDs from opposite sides pointing towards the solution and an optical sensor perpendicular to the light sources, taking multiple measurements per second. One blue LED is used for the detection of ASFV target in the blood sample, and one red LED is used to detect the internal positive control to verify the device's accuracy. Optical data is collected and analyzed by the device's processor circuit board using an algorithm to detect a sigmoidal curve from the optical measurements. After the detection chamber is analyzed, a result is posted on the E-ink display screen as “Positive, Negative, or “Invalid”. A positive result is detected when there is a change in slope that reaches a threshold enforced in the algorithm. A negative result is considered when the optical background noise does not meet this threshold in evaluating the change of slopes and when the internal positive control provides the expected signal. An invalid result appears, when the device is used outside of the listed operating conditions, malfunction from electronics, battery discharge, or internal positive control signal fails to verify a true negative result.

The described separation of blood components can also be used for other analytical methods such as for immunoassays, antibody tests, antigen tests, enzyme assays, molecular sequencing, protein sequencing and other analytical assays and methods for blood or liquid analysis.

Biological matter other than blood can take advantage of the described separation methods either by precipitating materials in the sample or because there may be blood components present in the sample such as fecal samples, urine samples, spit, tears, vaginal swabs, urethral swabs, nasal swabs, oral swabs, skin swabs or swabs or discharge from any parts of an organism as well as sample tissue from any parts of an organism.

Each of the individual variations described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the disclosure.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the disclosure. Also, any optional feature of the variations described May be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the disclosure (in which case what is present herein shall prevail). The referenced items are provided solely for their disclosure prior to the filing date of the present application. Nothing herein is to be construed as an admission that the present disclosure is not entitled to antedate such material by virtue of prior disclosure.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this disclosure belongs.

This disclosure is not intended to be limited to the scope of the particular forms set forth, but is intended to cover alternatives, modifications, and equivalents of the variations or embodiments described herein. Further, the scope of the disclosure fully encompasses other variations that may become obvious to those skilled in the art in view of this disclosure.

We claim:

1. A method for detecting nucleic acid targets in a liquid blood sample comprising:
mixing the blood sample with reagents, wherein the reagents comprise a divalent cation;
heating the mixed sample and reagents;
separating a resulting precipitate from a liquid supernatant; and
performing a subsequent nucleic acid amplification reaction using the liquid supernatant without any further purification, dilution, or treatment.

2. The method of claim 1, where the divalent cation is magnesium.

3. The method of claim 1, where the heating comprises heating between 80 and 99 degrees Celsius.

4. The method of claim 1, where the mixed sample and reagents comprise oligo nucleotides used in the subsequent amplification reaction.

5. The method of any of claim 1, where the reagents comprise magnesium sulfate from 3 mM to 5 mM, tris buffer at pH 8.8, 0.5M betaine, and oligonucleotides.

6. The method of claim 1, where the separating comprises using centrifugation or filtering.

7. The method of claim 1, where the reagents comprise dried or a combination of dried and liquid reagents.

8. The method of claim 1, where the nucleic acid amplification is isothermal.

9. A system for detecting nucleic acid targets in a sample comprising:
a mixing element for mixing the sample with reagents, wherein the reagents comprise a divalent cation;
a heating chamber for heating the mixed sample and reagents;
a separating element for separating the resulting precipitate from the liquid supernatant; and
at least one reaction element for performing a subsequent nucleic acid amplification reaction using the liquid supernatant without any further purification, dilution, or treatment.

10. The system of claim 9, where the sample comprises a blood sample.

11. The system of claim 9, where the divalent cation is magnesium.

12. The system of claim 9, where heating comprises heating between 80 and 99 degree Celsius.

13. The system of claim 9, where the mixed reagents comprise oligo nucleotides used in the subsequent amplification reaction.

14. The system of claim 9, where the reagents comprise magnesium sulfate from 3 mM to 5 mM, tris buffer at pH 8.8, 0.5M betaine, and oligonucleotides.

15. The system of claim 9, where the separating element comprises a centrifuge or filter.

16. The system of claim 9, where the reagents comprise dried or a combination of dried and liquid reagents.

17. The system of claim 9, where the nucleic acid amplification is isothermal.

18. The system of claim 9, further comprising a detection chamber for detecting a fluorescent signal indicative of the nucleic acid amplification.

19. The system of claim 9, where the sample is of oral, nasal, nasopharyngeal, throat, mouth, cheek, skin, a lesion, rectal, fecal, vaginal, or urethral origin.

20. A device for detecting nucleic acid targets in a blood sample comprising:
a reagent chamber,
an inlet port for receiving the liquid sample;
a processing chamber,
a processing chamber heater;
a processing chamber exit valve;
a processed sample filter for separating the supernatant from the precipitant;
a detection chamber, wherein the detection chamber is at least partially transparent;
an optical sensor adjacent to the detection chamber; and
a battery.
